(19) United States
(12) Patent Application Publication
(10) Pub. No.: US 2024/0261146 A1
(43) Pub. Date: Aug. 8, 2024

(54) METHOD FOR ADJUSTING OPTIMIZED RADIATION PARAMETERS OF LASER PULSES FOR AN OPHTHALMOLOGICAL LASER

(71) Applicant: SCHWIND EYE-TECH-SOLUTIONS GMBH, Kleinostheim (DE)

(72) Inventors: Samuel ARBA MOSQUERA, Aschaffenburg (DE); Pascal NAUBEREIT, Laufach (DE)

(21) Appl. No.: 18/432,535

(22) Filed: Feb. 5, 2024

(30) Foreign Application Priority Data
Feb. 8, 2023 (DE) 10 2023 103 036.1

(51) Int. Cl. A61F 9/008

(52) U.S. Cl. CPC A61F 9/00802

(57) ABSTRACT

The invention relates to a method for adjusting optimized irradiation parameters of laser pulses (24) for an ophthalmological laser (12) of a treatment apparatus (10), including, as steps, ascertaining (S10) a threshold value for a laser-induced optical breakthrough, wherein the threshold value is preset to a control device (18) of the treatment apparatus (10); providing (S12) an energy window of a laser pulse energy depending on the ascertained threshold value by the control device (18); selecting (S14) a laser pulse energy from the provided energy window; providing (S16) at least one spatial pulse distance range of the laser pulses (24) depending on the selected laser pulse energy by the control device (18), wherein the pulse distance range is determined based on the selected laser pulse energy by means of a pulse distance model; and selecting (S18) at least one spatial laser pulse distance (a, b) from the provided pulse distance range.

### FIELD

The invention relates to a method for adjusting optimized irradiation parameters of laser pulses for an ophthalmological laser of a treatment apparatus. Furthermore, the invention relates to a control device, which is formed to perform the method, to a treatment apparatus with an ophthalmological laser and such a control device, to a computer program including commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect an optical breakthrough, in particular a photodisruption and/or photoablation, in a focus situated within the organic tissue, to perform incisions in the cornea, for example to remove a tissue, in particular a tissue lenticule, from the cornea. Alternatively or additionally, ophthalmological lasers can be operated below an optical breakthrough threshold to achieve a characteristic change of the cornea, in particular a laser-induced refractive index change (LIRIC) or a cross-linking.

By focusing a laser pulse, non-linear absorption processes arise within the focus volume, which results in a very fast temperature and pressure increase in the form of a laser-induced optical breakthrough in the form of a plasma expansion upon exceeding a critical value. Herein, a shock wave arises, which propagates into the surrounding medium and causes the formation of a cavitation bubble, which allows the separation of the tissue. The high temperature and the high pressure of the gas in the bubble then result in the bubble oscillation, wherein this image applies to water and cannot be completely transferred to corneal tissue, in which the bubble expansion is limited against the restoring forces of the lamellar structure of the cornea. This has severe influences on intrastromal bubble dynamics, which finally results in a smaller bubble size in the cornea compared to water with identical pulse energy. Accordingly, an incision efficiency of the laser can be reduced and not be in an optimum range.

### SUMMARY

It is the object of the present invention to provide optimized irradiation parameters for an ophthalmological laser.

This object is solved by the method according to the invention, the devices according to the invention, the computer program according to the invention as well as the computer-readable medium according to the invention. Advantageous embodiments of the invention are specified in the respective dependent claims, the following description as well as the figures.

The invention is based on the idea to ascertain a laser pulse effect diameter, in particular a diameter of a cavitation bubble or a diameter of a laser-induced refractive index change, depending on a selected energy of the laser to thereby be able to adjust spatial laser pulse distances in optimized manner. Thus, the incision efficiency of the laser in the cornea can for example be optimized to the cavitation bubble diameter, such that the cavitation bubbles result in a contiguous incision, preferably such that the cavitation bubbles are not over-dimensioned and a thermal effect is limited, respectively.

By the invention, a method for adjusting optimized irradiation parameters of laser pulses for an ophthalmological laser of a treatment apparatus is provided. The following steps can for example be performed by a control device, wherein the control device can include a processor, in particular a microprocessor, which plans and/or performs one or more steps of the method.

In the method, ascertaining a threshold value for a laser-induced optical breakthrough, wherein the threshold value is preset to a control device of the treatment apparatus, providing at least one energy window including a selection of laser pulse energies depending on the ascertained threshold value by the control device and selecting a laser pulse energy from the provided energy window are effected. Furthermore, providing at least one spatial pulse distance range including a selection of laser pulse distances of the laser pulses depending on the selected laser pulse energy by the control device, wherein the pulse distance range is determined by means of a pulse distance model based on the selected laser pulse energy, and selecting at least one spatial laser pulse distance from the provided pulse distance range are effected.

In other words, a threshold value for a laser-induced optical breakthrough can first be provided for a control device of the ophthalmological laser, based on which the control device then determines at least one energy window, in which laser pulse energies are provided, at which the laser can efficiently operate. For example, an energy window can be provided above the threshold value for the laser-induced optical breakthrough, in which laser pulse energies for a cutting operation in the cornea are preset. Alternatively or additionally, an energy window can also be preset below the threshold value to for example provide laser pulse energies for a laser-induced optical refractive index change (LIRIC). Then, a laser pulse energy can be selected from the energy window, which is desired or intended for the treatment of the cornea. Herein, the selection can be manually effected by a user or the control device can select a laser pulse energy from the provided energy window in automated manner, for example depending on a planned treatment, which can be preset to the control device.

After selecting the laser pulse energy, the control device can ascertain a spatial pulse distance range, which is optimum for the selected laser pulse energy. This means that in the ascertained range for spatial pulse distances, only those pulse distances are offered, which are optimized for the selected laser pulse energy. Thus, for example for a laser disruption in the cornea, only those pulse distances can be provided, at which the cavitation bubbles still overlap each other and thus result in an optimized incision in the cornea. Herein, different pulse distances in the provided pulse distance range can include a degree of an overlap between adjacent pulses. In order to ascertain this pulse distance range, a pulse distance model can be used, by which the optimized laser pulse distances can be calculated. In particular, the control device can ascertain a laser pulse effect diameter, that is for example a cavitation bubble diameter, for the selected laser pulse energy in the cornea by means of the pulse distance model. Furthermore, pulse distances can be provided depending on the ascertained laser pulse effect diameter by the pulse distance model such that multiple laser pulse distances from a minimum intersection up to a maximum intersection are provided for selection. Minimum intersection means that edges of adjacent laser pulse effect diameters barely overlap each other and maximum intersection means that the centers of the laser pulse effects are close to each other except for a minimum distance, wherein further intermediate distances between these limits can be provided.

From this provided spatial pulse distance range, a spatial laser pulse distance can finally be selected, which is used for the treatment with the ophthalmological laser. In particular, control data, which includes a preset irradiation pattern for generating incision surfaces in the cornea, can be adapted by means of the selected spatial laser pulse distance to provide treatment positions or a laser pulse placement in the cornea, corresponding to the selected spatial laser pulse distance. This means, the control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

By the method, an optimized application of irradiation parameters for the laser-induced refractive index change can be determined on the one hand in that the energy window is provided for a range below the threshold value for the laser-induced optical breakthrough, or an optimized application of an incision in the cornea by means of optical breakthrough can be provided on the other hand in that the energy window is provided for a range above the threshold value for the laser-induced optical breakthrough. Preferably, the thus selected irradiation parameters can be validated for a treatment in that it is examined during the treatment that the irradiation parameters are complied with and/or it can be examined if the selected irradiation parameters are actually the optimized irradiation parameters for the respective treatment apparatus in that the irradiation parameters are varied in calibration measurements and the effect is examined.

Overall, the advantage arises that optimum ranges for a respective treatment are provided in automated manner, which improves a treatment with an ophthalmological laser.

The invention also includes embodiments, by which additional advantages arise.

In an embodiment the threshold value of the laser-induced optical breakthrough is measured. In other words, the threshold value, which is preset to the control device of the treatment apparatus, can be determined by measurements, in particular by measurement on an adequate material, by which a cornea can be simulated. For example, an artificial cornea and/or an animal cornea, for example of a pig, can be used. Thus, an advantageous configuration for determining the threshold value can be provided.

In a further embodiment the threshold value of the laser-induced optical breakthrough is calculated by the control device. Hereto, the formula

$${LIOB}_{Th} = C * \sqrt[3]{τ} * λ * \sqrt{m} * M {2}^{2} / ( {NA}^{2} * SR )$$

can be used for calculating the threshold value of the laser-induced optical breakthrough (LIOB_{Th}), wherein τ is the pulse length of the laser pulses, m is the number of photons, M2 is the quality factor of the laser beam, SR is the Strehl ratio, which indicates a quality factor of the optical beam path, λ is the wavelength and NA is the numerical aperture. C is a proportionality constant for the laser-induced optical breakthrough, which can for example be calculated or be determined by measurement. Hereby, the advantage arises that the threshold value of the laser-induced optical breakthrough can be fast determined for different adjustments of the ophthalmological laser without having to perform expensive measurements.

In a further embodiment the energy window of the laser pulse energy is calculated by multiplication or division of the range from 1.2 to 4 by the ascertained threshold value. In other words, energies are provided in the energy window, which include 1.2 times to four times the threshold value for the laser-induced optical breakthrough. Therein, the multiplication can preferably be used in ascertaining optimum incision parameters, and the division can be used in avoiding incisions, for example in the laser-induced refractive index change. The limits of this range are optimized since an optical breakthrough can thus be provided in secured manner on the one hand, in particular for the limit of 1.2, and the development of too large cavitation bubbles, in particular at the limit value of 4, which would be disadvantageous for the treatment of the cornea, can be avoided on the other hand.

In a further embodiment the laser pulse energies, which are given for selection for the energy window, are additionally provided by a preset incision criterion. This means that not only the threshold value of the laser-induced optical breakthrough presets, which laser pulse energies are given for selection in the energy window, but also a preset incision criterion. Therein, the incision criterion can be a condition or a rule, according to which the decision for the provided laser pulse energies can be made. In particular, a characteristic of the laser pulse effect diameter and thereby of the generated incision in the cornea can be preset based on the incision criterion. By the incision criterion, only such laser pulse energies can preferably be provided in the energy window, at which the development of an opaque bubble layer and/or of “black spots” is minimized. Alternatively or additionally, only such laser pulse energies can also be given for selection for the energy window by the incision criterion, at which an optimized incision or separation effect can be achieved according to predetermined objective or subjective evaluations. Herein, energy thresholds or energy ranges can for example be predetermined, which satisfy the incision criterion. By this development, the advantage arises that the provided laser pulse energies can be optimized.

In a further embodiment the pulse distance range is determined by means of a laser pulse effect diameter divided by preset overlap factors in the pulse distance model, wherein the laser pulse effect diameter is determined by means of d=K*(E_{Pulse}−LIOB_{Th})\hat{ }(⅓), wherein d is the laser pulse effect diameter, K is a tissue factor, E_{Pulse} is the selected energy and LIOB_{Th} is the threshold of the laser-induced optical breakthrough. In other words, the laser pulse distances, which are provided in the pulse distance range, result from the cube root of the respective energy portion, which is above the optical breakthrough threshold, multiplied by a tissue factor, which can in particular be predetermined for the cornea. The calculated laser pulse effect diameter can then be divided by the respective overlap factor (or be multiplied by a reciprocal value) to obtain a laser pulse distance for each of the preset overlap factors, which together result in the pulse distance range. Therein, the overlap factor can for example be selected in a range from 1 to 10, preferably in a range from 1 to 3. Furthermore, an additional offset term can also be provided for the pulse distance range, which is preset in a range from 0 to 10 micrometers, preferably 0 to 2 micrometers, and which presets an additional distance of adjacent laser pulses. A cavitation bubble diameter or for example an area, in which a characteristic change, in particular a laser-induced refractive index change, occurs, is meant by the laser pulse effect diameter. Hereby, a suitable configuration for determining optimized ranges for a spatial laser pulse distance can be calculated.

In a further embodiment the spatial pulse distance range includes a distance between adjacent laser pulses on a laser pulse path and/or a distance of laser pulse paths. In other words, the laser pulse distances, which are provided in the spatial pulse distance range, can include distances between subsequent laser pulses. Alternatively or additionally, the pulse distance range can also include distances between adjacent laser pulse paths, which are for example generated for generating a laser pulse pattern, in particular interfaces, in the cornea.

In a further embodiment pulse distances are provided for the spatial pulse distance range, for which a ratio between the distance of adjacent laser pulses on a laser pulse path and the distance of adjacent laser pulse paths is within predetermined limit values, in particular between 0.1 and 10, preferably between 0.2 and 5. In other words, preset limit values are provided, which set the relation of the distances of adjacent laser pulses divided by the distances of adjacent laser pulse paths. This means, it is to apply:

$${R}_{1} > {A}_{Laser pulse distance} / {A}_{Laser pulse path distance} < {R}_{2} ,$$

wherein R₁ is a lower limit value, in particular 0.1 or 0.2, A_{Laser pulse distance} is the distance of adjacent laser pulses on a laser pulse path, A_{Laser pulse path distance} is the distance of adjacent laser pulse paths and R₂ is an upper limit value, in particular 10 or preferably 5. Thus, suitable pulse distances can be provided, which are advantageous for a treatment of a cornea.

In a further embodiment the previously mentioned limit values are set depending on the energy window. This means that the limit values R₁ and R₂ for the pulse distances are selected in automated manner depending on the selected laser pulse energy. In particular, for laser pulse energies of 1.25 to 2.25, the lower limit R₁ can be selected at 0.25 and the upper limit R₂ at 2^{-1/2} or the lower limit R₁ at 2^{-1/2} and the upper limit R₂ at 4. For laser pulse energies of 2.25 to 4, the lower limit R₁ can be selected at (\sqrt{5}−1)/2 and the upper limit R₂ at (\sqrt{5}+1)/2. Thus, optimized ratios between the distance of adjacent laser pulses to the laser pulse paths can be provided depending on the laser pulse energy.

A further embodiment relates to a method for controlling a treatment apparatus. Therein, the method includes the method steps of at least one embodiment of a method as it was previously described. Furthermore, the method includes providing the irradiation parameters, thus at least the laser pulse energy and the laser pulse distance, as control data for controlling the treatment apparatus, wherein the treatment apparatus and/or the ophthalmological laser can be controlled by means of the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of the previously described method. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of the previously described method. The respective laser can be formed to perform respective incisions in a cornea by means of optical breakthrough, in particular to at least partially separate a predefined corneal volume with predefined interfaces of a human or animal eye by means of optical breakthrough, in particular to at least partially separate it by means of photodisruption and/or to ablate corneal layers by means of (photo)ablation and/or to effect a laser-induced refractive index change in the cornea and/or the eye lens.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiment of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts an exemplary representation of laser pulses on laser pulse paths for generating incision surfaces.

FIG. 3 depicts a schematic method diagram according to an exemplary embodiment.

In the figures, identical or functionally identical elements are provided with the same reference characters.

### DETAILED DESCRIPTION

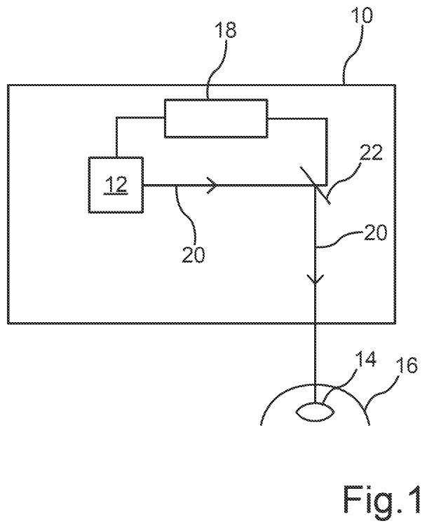

FIG. 1 shows a schematic representation of a treatment apparatus 10 with an ophthalmological laser 12 for the removal of a tissue 14 from a cornea 16 of a human or animal eye by means of photodisruption and/or photoablation or for a laser-induced refractive index change. For example, the tissue 14 can represent a lenticule or also volume body, which is to be separated out of the cornea 16 of the eye by the ophthalmological laser 12 for correcting a visual disorder. A geometry of the tissue 14 to be removed can be provided by a control device 18, in particular in the form of control data, such that the laser 12 emits pulsed laser pulses in a pattern predefined by the control data into the cornea 16 of the eye to remove the tissue 14. Alternatively, the control device 18 can be a control device 18 external with respect to the treatment apparatus 10.

Furthermore, FIG. 1 shows that the laser beam 20 generated by the laser 12 can be deflected towards the eye by means of a beam deflection means 22, namely a beam deflection device such as for example a rotation scanner, to remove the tissue 14. The beam deflection device 22 can also be controlled by the control device 18.

The illustrated laser 12 can preferably be a photodisruptive and/or photoablative laser, which is formed to emit laser pulses in a wavelength range between 300 nanometers and 1400 nanometers, preferably between 700 nanometers and 1200 nanometers, at a respective pulse duration between 1 femtosecond and 1 nanosecond, preferably between 10 femtoseconds and 10 picoseconds, and a repetition frequency of greater than 10 kilohertz, preferably between 100 kilohertz and 100 megahertz. Optionally, the control device 18 additionally comprises a storage device (not illustrated) for at least temporary storage of at least one control dataset, wherein the control dataset or datasets include(s) control data for positioning and/or for focusing individual laser pulses in the cornea.

Furthermore, the control device 18 can be formed to ascertain optimized irradiation parameters for treating the cornea 16, in particular for separating the tissue 14. Preferably, a laser pulse energy and the matching laser pulse distance of laser pulses of an irradiation pattern can be ascertained as the irradiation parameters. Hereto, a threshold value for a laser-induced optical breakthrough can be present to the control device 18, which can for example be determined by means of measurements on materials, which have similar characteristics as the cornea 16. Alternatively, the threshold value of the laser-induced optical breakthrough can be calculated by the control device 18 for the treatment apparatus 10 with the formula:

$${LIOB}_{Th} = C * \sqrt[3]{τ} * λ * \sqrt{m} * M {2}^{2} / ( {NA}^{2} * SR )$$

wherein LIOBₜₕ is the threshold value of the optical breakthrough, t is the pulse length, λ is the wavelength, m is the number of photons, M2 is the quality factor of the laser beam, SR is the Strehl ratio, NA is the numerical aperture and C is a proportionality constant.

By means of the threshold value of the laser-induced optical breakthrough, the control device 18 can then provide laser pulse energies, which are suitable for a treatment, wherein either an energy window in a range above the threshold value, in particular if incisions in the cornea 16 are intended, or an energy window below the threshold value, in particular if a laser-induced characteristic change in the tissue 14 is intended, can be provided thereto. According to selection of the laser pulse energy from the respective energy window, the control device 18 can then provide laser pulse distances, which are suitable for the selected laser pulse energy. Thereto, a pulse distance model can be provided, by which a laser pulse effect diameter for a respective laser pulse energy from the energy window is first calculated and respective laser pulse distances for the respectively ascertained laser pulse effect diameter are subsequently calculated. Therein, the laser pulse effect diameter is the diameter of the effect of the respective laser pulse in the cornea 16. Thus, the laser pulse effect diameter can for example be a cavitation bubble diameter or a diameter of an area, in which a characteristic change occurs. Preferably, the laser effect diameter can be calculated by means of the formula

$$d = K * ( {E}_{Pulse} - {LIOB}_{th} ) \hat{} ( 1 / 3 )$$

wherein d is the respective laser pulse effect diameter, K is a tissue factor, E_{Pulse} is the laser pulse energy and LIOBₜₕ is the threshold value of the optical breakthrough. By this formula, it becomes clear that the laser pulse effect diameter continuously increases with the energy portion, which is above the threshold of the optical breakthrough, whereby an optimized laser pulse distance for a treatment of the cornea 16 can thus be ascertained by the control device 18.

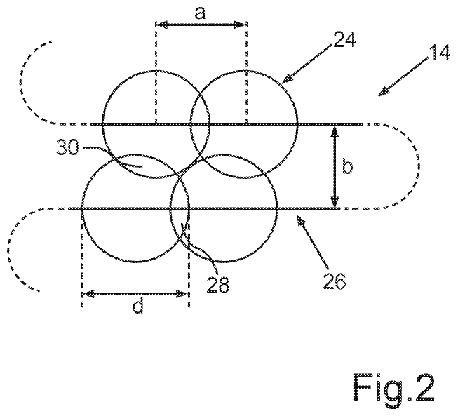

In FIG. 2, an exemplary representation of laser pulses 24 on laser pulse paths 26, for example for generating incision surfaces for removing the tissue 14, is illustrated. According to selection of the laser pulse energy, the laser pulse effect diameter d can herein increase, wherein an optimized distance a between adjacent laser pulses 24, which are situated on a laser pulse path 26, can thus be ascertained. Further, an overlap area 28 between adjacent laser pulses can preferably be adjusted by means of an overlap factor.

Alternatively or additionally, a distance of laser pulse paths b can be ascertained from the pulse distance model as the spatial laser pulse distance, wherein a further overlap factor for an overlap area 30 of laser pulses 24 of adjacent laser pulse paths 26 can be preset here too.

Therein, a ratio between the distance of adjacent laser pulses a and the distance of adjacent laser pulse paths b can preferably be preset such that they range within limit values between 0.1 and 10, preferably between 0.2 and 5. In particular, the limit values can be preset from the laser pulse energy, which is used, whereby the ratio of the distances a/b is automatically limited between predetermined limit values.

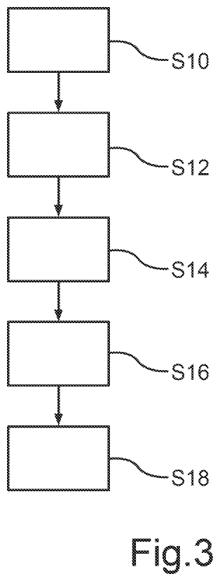

In FIG. 3, a schematic method diagram for adjusting optimized irradiation parameters of laser pulses 24 for an ophthalmological laser 12 of a treatment apparatus 10 is illustrated. In a step S10, a threshold value for a laser-induced optical breakthrough can be ascertained, preferably by the control device 18, which can calculate the threshold value depending on parameters of the treatment apparatus 10.

In a step S12, an energy window of laser pulse energies can then be provided by the control device 18 depending on the threshold value, which are suitable for a treatment of the cornea 16 considering the threshold value. Therein, the energy window can particularly preferably comprise laser pulse energies, which are multiplied by the threshold value with a factor of 1.2 to 4.

In a step S14, a laser pulse energy can then be selected from the provided energy window, wherein the selection can for example be performed by a user, who defines a desired laser pulse energy from the energy window.

Subsequently, the control device 18 can ascertain spatial laser pulse distances a and/or b with the aid of the selected laser pulse energy in a step S16, which can be provided for selection in the form of a spatial pulse distance range. Therein, the laser pulse distances can be ascertained by means of a pulse distance model, in which a laser pulse effect diameter d is ascertained from the energy difference between the selected laser pulse energy and the threshold value and considering a tissue factor. Furthermore, overlap factors can be provided, by which a respective overlap area 28, 30 between adjacent laser pulses 24 and/or adjacent laser pulse paths 26 can be provided.

Finally, spatial laser pulse distances a, b can be selected from the provided pulse distance range in a step S18, which are used for the treatment of the cornea 16, wherein the spatial laser pulse distances a, b are optimized to the selected laser pulse energy by means of the method.

Overall, the examples show, how an analytic optimization of a laser pulse treatment can be provided for an ophthalmological laser by the invention.

1. A method for adjusting optimized irradiation parameters of laser pulses for an ophthalmological laser of a treatment apparatus, comprising the steps:
ascertaining a threshold value for a laser-induced optical breakthrough, wherein the threshold value is preset to a control device of the treatment apparatus;
providing at least one energy window including a selection of laser pulse energies depending on the ascertained threshold value by the control device;
selecting a laser pulse energy from the provided energy window;
providing at least one spatial pulse distance range including a selection of laser pulse distances of the laser pulses depending on the selected laser pulse energy by the control device, wherein the pulse distance range is determined by means of a pulse distance model based on the selected laser pulse energy; and
selecting at least one spatial laser pulse distance from the provided pulse distance range.

2. The method according to claim 1, wherein the threshold value of the laser-induced optical breakthrough is measured.

3. The method according to claim 1, wherein the threshold value of the laser-induced optical breakthrough is calculated by the control device.

4. The method according to claim 1, wherein the energy window of the laser pulse energy is calculated by multiplication or division of the range from 1.2 to 4 by the ascertained threshold value.

5. The method according to claim 1, wherein the laser pulse energies, which are given for selection for the energy window, are additionally provided by a preset incision criterion.

6. The method according to claim 1, wherein the pulse distance range is determined by means of a laser pulse effect diameter divided by preset overlap factors in the pulse distance model, wherein the laser pulse effect diameter is determined by means of d=K*(E_{Pulse}−LIOBₜₕ)\hat{ }(⅓), wherein d is the laser pulse effect diameter, K is a tissue factor, E_{Pulse} is the selected laser pulse energy and LIOBₜₕ is the threshold of the laser-induced optical breakthrough.

7. The method according to claim 6, wherein the overlap factors include values from 1 to 10.

8. The method according to claim 1, wherein the spatial pulse distance range includes a distance between adjacent laser pulses on a laser pulse path and/or a distance of laser pulse paths.

9. The method according to claim 8, wherein pulse distances are provided for the spatial pulse distance range, for which a ratio between the distance of adjacent laser pulses on a laser pulse path and the distance of adjacent laser pulse paths is within predetermined limit values, in particular between 0.1 and 10, preferably between 0.2 and 5.

10. The method according to claim 9, wherein the limit values are set depending on the energy window.

11. A control device, which is configured to perform a respective method according to claim 1.

12. A treatment apparatus with at least one eye surgical laser for separation of a corneal volume with predefined interfaces of a human or animal eye by means of optical breakthrough, in particular by means of photodisruption and/or photoablation, and at least one control device according to claim 11.

13. A non-transitory computer-readable medium configured for storing a computer program, the computer program including commands which cause a treatment apparatus to execute the method according to claim 1.

14. (canceled)
